(19) United States
(12) Patent Application Publication
(10) Pub. No.: US 2004/0116759 A1
(43) Pub. Date: Jun. 17, 2004

(54) Oligomerization of hydrocarbons

(76) Inventors: Bruce B. Randolph, Bartlesville, OK (US); Fan-Nan Lin, Bartlesville, OK (US); Linda D. Williams, Bartlesville, OK (US)

Correspondence Address: RICHMOND, HITCHCOCK,, FISH & DOLLAR, P.O. Box 2443, Bartlesville, OK, 74005, US

(21) Appl. No.: 10/319,726

(22) Filed: Dec. 13, 2002

(51) Int. Cl. C07C 2/02

(52) U.S. Cl. 585/531

(57) ABSTRACT

A novel oligomerization process is provided and includes contacting a hydrocarbon feedstock comprising at least one olefin with an oligomerization catalyst comprising a support component, a metal, and a halogen in an oligomerization reaction zone under oligomerization reaction conditions.

### BACKGROUND OF THE INVENTION

The present invention relates to the field of hydrocarbon upgrading processes. In another aspect, the invention relates to the oligomerization of hydrocarbons.

It is known in the art to oligomerize hydrocarbons, such as olefins, in the presence of a catalyst to diesel fuel range hydrocarbons. However, production of high quality from the oligomerization of hydrocarbons with a paraffin isomerization catalyst is unexpected. Therefore, development of a process for oligomerizing hydrocarbons to gasoline range hydrocarbons wherein the quality of the hydrocarbon produced is enhanced would be a significant contribution to the art and to the economy.

### SUMMARY OF THE INVENTION

It is an object of the present invention to provide a process for oligomerizing hydrocarbons to gasoline range hydrocarbons or diesel range hydrocarbons, depending upon the boiling point of the feedstock.

It is yet another object of the present invention to provide a process for oligomerizing hydrocarbons utilizing a catalyst comprising a support component, a metal, and a halogen.

It is still another object of the present invention to provide a process for oligomerizing hydrocarbons utilizing a catalyst comprising a support component, a metal, and a halogen, and heating the catalyst in the presence of hydrogen before oligomerization.

In accordance with the present invention, a process has been discovered which comprises, consists of, or consists essentially of contacting a hydrocarbon feedstock comprising at least one olefin with a catalyst comprising:

(a) a support component

(b) a metal selected from the group consisting of platinum, palladium, iron, cobalt, zinc, nickel, ruthenium, rhodium, osmium, iridium, and combinations of any two or more thereof, and

(c) a halogen

in an oligomerization reaction zone under oligomerization reaction conditions.

The second embodiment of the invention is a process which comprises, consists of, or consists essentially of:

(1) heating a catalyst, wherein the catalyst comprises:

(a) a support component;

(b) a metal selected from the group consisting of platinum, palladium, iron, cobalt, zinc, nickel, ruthenium, rhodium, osmium, iridium and combinations of any two or more thereof; and

(c) a halogen;

(2) contacting the catalyst with a gas during the heating, and

(3) contacting a hydrocarbon feedstock comprising at least one olefin with the catalyst in an oligomerization reaction zone under oligomerization reaction conditions.

Other objects and advantages will become apparent from the detailed description and the appended claims.

### DETAILED DESCRIPTION OF THE INVENTION

The term “hydrocarbon feedstock” is generally referred to, unless otherwise indicated, as one or more hydrocarbons or substituted hydrocarbons having in the range of from 2 carbon atoms to 30 carbon atoms per molecule, preferably 2 to 20 carbon atoms per molecule, and more preferably 2 to 16 carbon atoms per molecule. Non-limiting examples of suitable hydrocarbon feedstocks include gasolines from catalytic oil cracking (e.g., FCC and hydrocracking) processes, pyrolysis gasolines from thermal hydrocarbon (e.g., ethane, propane, and naphtha) cracking processes, coker naphtha, light coker naphtha, straight run naphtha, light naphtha and the like. The most preferred hydrocarbon feedstock comprises at least one olefin containing in the range of from 2 to 10 carbon atoms per molecule.

The catalyst useful in the present invention comprises, consists of, or consists essentially of

(a) a support component,

(b) a metal selected from the group consisting of platinum, palladium, iron, cobalt, zinc, nickel, ruthenium, rhodium, osmium, iridium, and combinations of any two or more thereof, and

(c) a halogen.

Preferably, the halogen is selected from the group consisting of chlorine and bromine. The metal is selected from the group consisting of platinum, palladium, and combinations thereof, and the support component is selected from the group consisting of alumina, silica-alumina, a zeolite, zirconia, a borate, an aluminum borate, and combinations thereof. Most preferably, the support component comprises alumina, the metal comprises platinum, and the halogen comprises chlorine.

Preferably, the hydrocarbon stream comprises at least one olefin having in the range of from 2 to 10 carbon atoms per molecule. More preferably, the hydrocarbon stream contains at least one olefin having in the range of from 2 to 6 carbon atoms per molecule. Most preferably, the hydrocarbon stream contains at least one olefin having in the range of from 5 to 6 carbon atoms per molecule. In addition, the hydrocarbon stream can be obtained by fractionation of a gasoline stream, including, but not limited to, fluidized catalytic cracker gasoline, to at least partially remove the C₅/C₆ olefin material for use as the hydrocarbon stream in the present invention.

The oligomerization reaction takes place in an oligomerization reaction zone. The oligomerization reaction zone can be any reactor system known to those skilled in the art to be suitable for use in oligomerizing an olefin in the presence of an oligomerization catalyst. Typical reactor systems useful in the present invention include, but are not limited to, batch type operations, a fixed bed system, a moving bed system, and a fluidized bed system.

Any of these operational modes has advantages and disadvantages, and those skilled in the art can select the one most suitable for a particular feed and catalyst system.

The oligomerization reaction is carried out within an oligomerization reaction zone, wherein is contained an oligomerization catalyst in accordance with the present invention, and under oligomerization reaction conditions that suitably promote oligomerization of at least a portion of the olefins of the hydrocarbon stream. Optionally, the oligomerization can take place in the presence of hydrogen. Optionally, an organic chloride can be charged to the oligomerization reaction zone. The oligomerization reaction conditions include a reaction temperature in the range of from about 100° F. to about 500° F., preferably in the range of from about 150° F. to about 300° F., and most preferably in the range of from 200° F. to, 260° F. The oligomerization reaction conditions include a pressure sufficient to maintain the reactants in liquid form. The contacting pressure of the oligomerization reaction zone is generally within the range of from about 0 psig to about 1000 psig, preferably in the range of from about 50 psig to about 500 psig, and most preferably from 100 psig to 250 psig.

The flow rate at which the hydrocarbon feedstock is charged to the oligomerization zone is such as to provide a weight hourly space velocity (“WHSV”) in the range of from about 0.01 to about 1000 hours⁻¹. The term “weight hourly space velocity”, as used herein, denotes the numerical ratio of the rate at which the hydrocarbon feedstock is charged to the oligomerization reaction zone in grams per hour divided by the grams of catalyst contained in the oligomerization reaction zone to which the hydrocarbon feedstock is charged. The preferred WHSV of the hydrocarbon feedstock to the oligomerization reaction zone is preferably in the range of from about 0.25 hour⁻¹ to about 250 hour⁻¹ and, most preferably, in the range of from 0.5 hour⁻¹ to 100 hour⁻¹.

The reactor effluent can be separated in a separation unit to produce a light product stream comprising C₆-C₉ components, and a heavy product stream comprising C₁₀⁺ components (components with 10 or more carbon atoms per molecule), primarily comprising C₁₀⁺ olefins.

In another embodiment of this invention, the catalyst is heated prior to being contacted with the hydrocarbon feedstock.

The catalyst is generally heated to a temperature in the range of from about 100° F. to about 500° F. Preferably, the catalyst is heated to a temperature in the range of from about 250° F. to about 400° F. More preferably, the catalyst is heated to a temperature in the range of from 300° F. to 360° F.

The catalyst is generally heated for a time period of at least 10 hours. Preferably, the catalyst is heated for a time period of at least 15 hours.

During the heating, the catalyst can be contacted with a gas. The gas is selected from the group consisting of hydrogen and nitrogen. Generally, the catalyst is contacted with a gas at a rate in the range of from about 0.01 ft³/hr to about 20 ft³/hr. Preferably, the catalyst is contacted with a gas at a rate in the range of from about 0.05 ft³/hr to about 10 ft³/hr. More preferably, the catalyst is contacted with a gas at a rate in the range of from 0.15 ft³/hr to 2 ft³/hr.

The catalyst is then contacted with a hydrocarbon feedstock in an oligomerization reaction zone under oligomerization reaction conditions, as described above.

The following examples are presented to further illustrate the invention and are not to be construed as unduly limiting its scope.

### EXAMPLE I

A 20 mL sample of platinum on chlorided alumina catalyst (UOP I-8) was placed into a tubular reactor with an inert support above and below the catalyst. A feed comprising about 50-60 weight percent isopentane, and about 40-50 weight percent pentene isomers was charged to the reactor at a feed rate of 20 mL/hr (LHSV=2 hr⁻¹). The initial temperature was set at 350° F. and no hydrogen/chloride co-feeds were used. The results are shown in Table I. Note that the columns Prod 1, Prod 2, Prod 3, Prod 4, Prod 5, and Prod 6 represent product samples taken after 1, 2, 3, 4, 5 and 6 hours on stream, respectively.

TABLE I
Oligomerization With Platinum on Chlorided Alumina Catalyst With No Pretreatment

| | Feed | Prod 1 | Prod 2 | Prod 3 | Prod 4 | Prod 5 | Prod 6 |
|---|---|---|---|---|---|---|---|
| Component (wt %) | | | | | | | |
| Ethane | 0.000 | 0.010 | 0.001 | 0.000 | 0.005 | 0.003 | 0.000 |
| Propane | 0.000 | 0.164 | 0.022 | 0.007 | 0.027 | 0.020 | 0.003 |
| Isobutane | 0.034 | 6.076 | 0.637 | 0.118 | 0.224 | 0.068 | 0.019 |
| Isobutene | 0.000 | 0.024 | 0.242 | 0.182 | 0.051 | 0.000 | 0.021 |
| Normal Butane | 0.098 | 0.713 | 0.057 | 0.047 | 0.007 | 0.045 | 0.044 |
| Trans-2-butene | 0.000 | 0.184 | 0.126 | 0.114 | 0.112 | 0.113 | 0.111 |
| Cis-2-butene | 0.000 | 0.012 | 0.028 | 0.019 | 0.044 | 0.013 | 0.005 |
| Neo-pentane | 0.000 | 0.020 | 0.031 | 0.003 | 0.001 | 0.002 | 0.003 |
| 3-methylbutene-1 | 0.000 | 0.012 | 0.080 | 0.056 | 0.100 | 0.034 | 0.030 |
| Isopentane | 50.603 | 75.691 | 58.481 | 51.533 | 50.261 | 49.918 | 49.826 |
| 1-pentene | 24.987 | 0.554 | 6.853 | 16.613 | 13.073 | 22.312 | 23.301 |
| 2-methlbutene-1 | 1.509 | 0.088 | 1.639 | 3.290 | 3.717 | 4.050 | 4.104 |
| Normal Pentene | 0.272 | 1.734 | 0.644 | 0.405 | 0.372 | 0.293 | 0.283 |
| Trans-2-pentene | 0.000 | 1.894 | 6.622 | 3.740 | 4.147 | 1.383 | 0.984 |
| Cis-2-pentene | 0.000 | 0.773 | 4.054 | 3.103 | 8.023 | 1.750 | 1.044 |
| 2-methylbutene-2 | 22.339 | 0.562 | 8.532 | 15.590 | 18.041 | 19.063 | 19.265 |
| Unknown C₁-C₅ | 0.000 | 0.011 | 0.011 | 0.013 | 0.005 | 0.005 | 0.075 |
| C₆-C₈ Napht/Olefins | 0.158 | 7.173 | 4.150 | 2.162 | 1.009 | 0.582 | 0.637 |
| C₉ | 0.000 | 2.977 | 4.099 | 1.398 | 0.230 | 0.089 | 0.043 |
| C₁₀⁺ | 0.000 | 1.326 | 3.691 | 1.605 | 0.551 | 0.256 | 0.206 |
| Conversion (%) | | | | | | | |
| 1-pentene Conversion | — | 97.783 | 72.575 | 33.511 | 47.681 | 10.704 | 6.747 |
| Isopentene Conversion | — | 97.221 | 57.012 | 20.595 | 8.342 | 2.936 | 1.879 |
| Normal Pentene Conversion | — | 87.108 | 29.846 | 6.123 | −1.025 | −1.836 | −1.369 |
| Total C₅ = Conversion | — | 92.046 | 43.112 | 13.190 | 3.549 | 0.494 | 0.217 |
| Total Olefin Conversion | — | 91.595 | 42.301 | 12.547 | 3.125 | 0.235 | −0.062 |

Note that the negative conversion is associated with the skeletal isomerization of olefins. While the catalyst's oligomerization begins to subside, the isomerization sites of the catalyst are still active.

### EXAMPLE II

A 20 mL sample of platinum on chlorided alumina catalyst (UOP I-8) was placed into a tubular reactor with an inert support above and below the catalyst. Prior to feed introduction, the catalyst was heated to 350° F. under a hydrogen flow rate of 1.5 ft³/hr for 18 hours. At that point, the temperature was reduced to 250° F. and a feed comprising of about 50-60 weight percent isopentane, and about 40-50 weight percent pentene isomers was charged to the reactor. A hydrogen co-feed was charged to the reactor at a rate of 50 cm³/min and perchloroethylene (PCE) was also introduced at a rate of 4 μL/hr. The results are shown in Table II. Note that the columns Prod 1, Prod 2, Prod 3, Prod 4, Prod 5, Prod 6, and Prod 7 represent product samples taken after 1, 2, 3, 4, 5, 6, and 7 hours on stream, respectively.

TABLE II
Oligomerization With Platinum on Chlorided Alumina
Catalyst With Catalyst Pretreatment

| | Feed | Prod 1 | Prod 2 | Prod 3 | Prod 4 | Prod 5 | Prod 6 | Prod 7 |
|---|---|---|---|---|---|---|---|---|
| Component (w % t) | | | | | | | | |
| Ethane | 0.000 | 0.041 | 0.003 | 0.000 | 0.000 | 0.000 | 0.000 | 0.000 |
| Propane | 0.000 | 0.183 | 0.011 | 0.002 | 0.002 | 0.001 | 0.000 | 0.000 |
| Isobutane | 0.034 | 22.724 | 2.395 | 0.233 | 0.127 | 0.109 | 0.087 | 0.049 |
| Isobutene | 0.000 | 0.003 | 0.010 | 0.022 | 0.027 | 0.036 | 0.041 | 0.043 |
| Normal Butane | 0.098 | 1.147 | 0.081 | 0.048 | 0.043 | 0.043 | 0.042 | 0.040 |
| Trans-2-butene | 0.000 | 0.165 | 0.126 | 0.123 | 0.115 | 0.115 | 0.112 | 0.109 |
| Cis-2-butene | 0.000 | 0.000 | 0.001 | 0.002 | 0.002 | 0.002 | 0.002 | 0.001 |
| Neo-pentane | 0.000 | 0.001 | 0.000 | 0.000 | 0.000 | 0.000 | 0.000 | 0.000 |
| 3-methylbutene-1 | 0.000 | 0.000 | 0.002 | 0.007 | 0.010 | 0.015 | 0.025 | 0.046 |
| Isopentane | 50.603 | 45.416 | 59.505 | 62.827 | 63.054 | 61.948 | 58.827 | 56.071 |
| 1-pentene | 24.987 | 0.007 | 0.055 | 0.126 | 0.076 | 0.091 | 0.193 | 0.330 |
| 2-methlbutene-1 | 1.509 | 0.003 | 0.036 | 0.111 | 0.151 | 0.264 | 0.484 | 0.999 |
| Normal Pentane | 0.272 | 19.388 | 23.132 | 22.240 | 24.803 | 24.321 | 21.241 | 17.832 |
| Trans-2-pentene | 0.000 | 0.003 | 1.066 | 2.370 | 1.417 | 1.687 | 3.349 | 5.456 |
| Cis-2-pentene | 0.000 | 0.000 | 0.312 | 0.690 | 0.414 | 0.493 | 0.992 | 1.647 |
| 2-methylbutene-2 | 22.339 | 0.005 | 0.297 | 0.852 | 1.130 | 1.889 | 3.457 | 7.074 |
| Unknown C₁-C₅ | 0.000 | 0.003 | 0.009 | 0.009 | 0.009 | 0.009 | 0.009 | 0.007 |
| 2,2-dimethylbutane | 0.000 | 1.177 | 0.082 | 0.005 | 0.005 | 0.000 | 0.000 | 0.000 |
| 2,3-dimethylbutane | 0.000 | 1.164 | 0.485 | 0.058 | 0.034 | 0.032 | 0.031 | 0.000 |
| 2-methylpentane | 0.000 | 3.560 | 1.650 | 0.166 | 0.078 | 0.067 | 0.054 | 0.023 |
| 2-methylpentane | 0.000 | 1.855 | 0.947 | 0.132 | 0.065 | 0.060 | 0.050 | 0.020 |
| Normal Hexane | 0.000 | 0.731 | 0.186 | 0.039 | 0.030 | 0.031 | 0.035 | 0.016 |
| Other C₆-C₈ | 0.158 | 2.195 | 2.192 | 1.158 | 0.922 | 0.951 | 1.104 | 1.118 |
| C₉ | 0.000 | 0.165 | 3.197 | 3.045 | 2.520 | 2.586 | 3.183 | 3.306 |
| C₁₀⁺ | 0.000 | 0.063 | 4.221 | 5.735 | 4.966 | 5.250 | 6.684 | 5.812 |
| Conversion (%) | | | | | | | | |
| 1-pentene | | 99.974 | 99.779 | 99.497 | 99.697 | 99.637 | 99.226 | 98.679 |
| Conversion | | | | | | | | |
| Isopentane | | 99.966 | 98.592 | 95.930 | 94.587 | 90.908 | 83.369 | 65.953 |
| Conversion | | | | | | | | |
| Normal Pentene | | 99.961 | 94.264 | 87.251 | 92.371 | 90.911 | 81.851 | 70.252 |
| Conversion | | | | | | | | |
| Total Pentene | | 99.963 | 96.378 | 91.489 | 93.453 | 90.910 | 82.593 | 68.153 |
| Conversion | | | | | | | | |
| Total Olefin | | 99.618 | 96.098 | 91.189 | 93.159 | 90.597 | 82.278 | 67.838 |
| Conversion | | | | | | | | |

Comparing Table II to Table I, it should be noted that the catalyst without the pre-treatment, as prepared in Example I, experiences a much higher drop in total olefin conversion than the oligomerization process with the catalyst pre-treatment in Example II.

### EXAMPLE III

A portion of the C₁₀⁺ products from Examples I and II (predominantly in the C₁₀ to C₁₆ region) were isolated and hydrogenated using 0.75 weight percent palladium on ⅛ inch Al₂O₃ spheres at a temperature of 350° F. The hydrogenated product was then analyzed by gc/ms. The average mass spectrum of the C₁₀-C₁₂ range components were consistent with for methylundecane and the C₁₃⁺ components were consistent with tetramethylheptadecane indicating saturation of the double bonds. A 5 lv % solution of the hydrogenated product of Example II was blended into isooctane. The resulting RON of the blend was determined to be 98.4 by standard engine tests. Based on linear blending rules, this material has a blending RON value of 68.

A 10 lv % solution of the C₁₀⁺ products that had not been hydrogenated was blended into isooctane. The resulting RON value of the blend was 99.5 as determined by standard engine tests. A second mixture was blended into isooctane with the resulting octane being 98.8. Assuming linear blending rules, the first mixture has a blending RON value of 95 and the second mixture has a blending RON value of 88.

Therefore, the C₁₀⁺ product of the inventive process has a high RON value and can be used as a gasoline blendstock.

Whereas this invention has been described in terms of the preferred embodiments, reasonable variations and modifications are possible by those skilled in the art. Such modifications are within the scope of the described invention and appended claims.

That which is claimed:

1. A process comprising contacting a hydrocarbon feed comprising at least one olefin with a catalyst comprising:
(a) a support component;
(b) a metal selected from the group consisting of platinum, palladium, iron, cobalt, zinc, nickel, ruthenium, rhodium, osmium, iridium, and combinations of any two or more thereof, and
(c) a halogen
in an oligomerization reaction zone under oligomerization reaction conditions.

2. A process in accordance with claim 1 wherein said at least one olefin has in the range of from 2 to 10 carbon atoms per molecule.

3. A process in accordance with claim 1 wherein said at least one olefin has in the range of from 2 to 6 carbon atoms per molecule.

4. A process in accordance with claim 1 wherein said at least one olefin has in the range of from 5 to 6 carbon atoms per molecule.

5. A process in accordance with claim 1 wherein said oligomerization reaction conditions include a temperature in the range of from about 100° F. to about 500° F.

6. A process in accordance with claim 1 wherein said oligomerization reaction conditions include a temperature in the range of from about 150° F. to about 300° F.

7. A process in accordance with claim 1 wherein said oligomerization reaction conditions include a temperature in the range of from 200° F. to 260° F.

8. A process in accordance with claim 1 further comprising reactivating said catalyst by stripping said catalyst with hydrogen.

9. A process in accordance with claim 1 wherein said metal of said catalyst is selected from the group consisting of platinum, palladium, and combinations thereof.

10. A process in accordance with claim 1 wherein said halogen of said catalyst is selected from the group consisting of chlorine, bromine and combinations thereof.

11. A process in accordance with claim 1 wherein said metal of said catalyst comprises platinum, said halogen comprises chlorine and said support component comprises alumina.

12. A process in accordance with claim 1 wherein an organic chloride is charged to said oligomerization reaction zone.

13. A process in accordance with claim 1 wherein said catalyst is heated prior to contacting with said hydrocarbon feed.

14. A process in accordance with claim 13 wherein said catalyst is contacted with a gas during said heating.

15. A process in accordance with claim 13 wherein said catalyst is heated to a temperature in the range of from about 100° F. to about 500° F.

16. A process in accordance with claim 13 wherein said catalyst is heated to a temperature in the range of from about 250° F. to about 400° F.

17. A process in accordance with claim 13 wherein said catalyst is heated to a temperature in the range of from 300° F. to 360° F.

18. A process in accordance with claim 13 wherein said catalyst is heated for a time period of at least 10 hours.

19. A process in accordance with claim 13 wherein said catalyst is heated for a time period of at least 15 hours.

20. A process in accordance with claim 14 wherein said gas comprises hydrogen.

21. A process in accordance with claim 14 wherein said gas comprise nitrogen.

22. A process in accordance with claim 14 wherein said catalyst is contacted with a gas at a rate in the range of from about 0.01 ft³/hr to about 20 ft³/hr hydrogen.

23. A process in accordance with claim 14 wherein said catalyst is contacted with a gas at a rate in the range of from about 0.05 ft³/hr to about 10 ft³/hr.

24. A process in accordance with claim 14 wherein said catalyst is contacted with a gas at a rate in the range of from 0.15 ft³/hr to 2 ft³/hr.

25. A process comprising:
(1) heating a catalyst, wherein said catalyst comprises
(a) a support component
(b) a metal selected from the group consisting of platinum, palladium, iron, cobalt, zinc, nickel, ruthenium, rhodium, osmium, indium and combinations of any two or more thereof, and
(c) a halogen,
(2) contacting said catalyst with a gas during said heating, and
(3) contacting a hydrocarbon feed comprising at least one olefin with said catalyst in an oligomerization zone under oligomerization reaction conditions.

26. A process in accordance with claim 25 wherein said at least one olefin comprises an olefin having in the range of from 2 to 10 carbon atoms per molecule.

27. A process in accordance with claim 25 wherein said at least one olefin has in the range of from 2 to 6 carbon atoms per molecule.

28. A process in accordance with claim 25 wherein said at least one olefin has in the range of from 5 to 6 carbon atoms per molecule.

29. A process in accordance with claim 25 wherein said oligomerization reaction conditions include a temperature in the range of from about 100° F. to about 500° F.

30. A process in accordance with claim 25 wherein said oligomerization reaction conditions include a temperature in the range of from about 150° F. to about 300° F.

31. Process in accordance with claim 25 wherein said oligomerization reaction conditions include a temperature in the range of from 200° F. to 260° F.

32. A process in accordance with claim 25 further comprising reactivating said catalyst by stripping said catalyst with hydrogen.

33. A process in accordance with claim 25 wherein said metal of said catalyst is selected from the group consisting of platinum, palladium, and combinations thereof.

34. A process in accordance with claim 25 wherein said halogen of said catalyst is selected from the group consisting of chlorine, bromine and combinations thereof.

35. A process in accordance with claim 25 wherein said metal of said catalyst comprises platinum, said halogen comprises chlorine and said support component comprises alumina.

36. A process in accordance with claim 25 wherein said catalyst is heated to a temperature in the range of from about 100° F. to about 500° F.

37. A process in accordance with claim 25 wherein said catalyst is heated to a temperature in the range of from about 250° F. to about 400° F.

38. A process in accordance with claim 25 wherein said catalyst is heated to a temperature in the range of from 300° F. to 360° F.

39. A process in accordance with claim 25 wherein said catalyst is heated for a time period of at least 10 hours.

40. A process in accordance with claim 25 wherein said catalyst is heated for a time period of at least 15 hours.

41. A process in accordance with claim 25 wherein said gas in step (2) comprises hydrogen.

42. A process in accordance with claim 25 wherein said gas in step (2) comprises nitrogen.

43. A process in accordance with claim 25 wherein said catalyst is contacted with a gas at a rate in the range of from about 0.01 ft³/hr to about 20 ft³/hr hydrogen.

44. A process in accordance with claim 25 wherein said catalyst is contacted with a gas at a rate in the range of from about 0.05 ft³/hr to about 10 ft³/hr.

45. A process in accordance with claim 25 wherein said catalyst is contacted with a gas at a rate in the range of from 0. 1 ft³/hr to 2 ft³/hr.

46. A process in accordance with claim 25 wherein an organic chloride is charged to said oligomerization reaction zone during step (3).
